(19) United States
(12) Patent Application Publication
(10) Pub. No.: US 2023/0285707 A1
(43) Pub. Date: Sep. 14, 2023

(54) AIR CIRCUIT FOR A RESPIRATORY THERAPY SYSTEM

(71) Applicant: ResMed Asia Pte. Ltd., Singapore (SG)

(72) Inventors: Jing CHEN, Singapore (SG); Teong Hong CHUAH, Singapore (SG); Marvin Sugi HARTONO, Singapore (SG)

(21) Appl. No.: 18/118,577

(22) Filed: Mar. 7, 2023

(30) Foreign Application Priority Data
Mar. 9, 2022 (AU) 2022900570

(51) Int. Cl. A61M 16/08; A61M 16/10

(52) U.S. Cl. CPC A61M 16/0816; A61M 16/10; A61M 2205/36

(57) ABSTRACT

An air circuit for a respiratory therapy system, the air circuit includes a conduit; and an electromagnet provided to at least a first end of the conduit. At least a portion of the conduit is collapsible and/or compressible in an axial direction, and the portion is adjacent the electromagnet. A helical wire of magnetic material is provided to a wall of the collapsible and/or compressible portion of the conduit. Energising the electromagnet causes at least a portion of the helical wire adjacent the electromagnet to be attracted to the electromagnet, thereby reducing the length of the conduit.

### 1 BACKGROUND OF THE TECHNOLOGY

### 1.1 Field of the Technology

The present technology relates to one or more of the screening, diagnosis, monitoring, treatment, prevention and amelioration of respiratory-related disorders. The present technology also relates to medical devices or apparatus, and their use.

### 1.2 Description of the Related Art

### 1.2.1 Human Respiratory System and Its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hyperventilation Syndrome (OHS). Chronic Obstructive Pulmonary Disease (COPD), Neuromuscular Disease (NMD) and Chest wall disorders.

### 1.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 1.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

### 1.2.3.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient’s face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the patient interface may not include a seal sufficient to facilitate delivery to the airways of a supply of gas at a positive pressure of about 10 cmH₂O. For flow therapies such as nasal HFT, the patient interface is configured to insufflate the nares but specifically to avoid a complete seal. One example of such a patient interface is a nasal cannula.

### 1.2.3.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

The designer of a device may be presented with an infinite number of choices to make. Design criteria often conflict, meaning that certain design choices are far from routine or inevitable. Furthermore, the comfort and efficacy of certain aspects may be highly sensitive to small, subtle changes in one or more parameters.

### 1.2.3.3 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

Air circuits for connection between an RPT and a patient interface are typically supplied in lengths of around 2 m. However, some patients may prefer a longer air circuit while others may find this length to be inconveniently long, and may prefer a shorter air circuit.

Air circuits which are sufficiently long to suit a majority of patients’ needs may be inconvenient to package, particularly since the need to provide reinforcing to the tube (to prevent crushing) may result in the tube being resistant to being held in a tightly coiled configuration.

### 1.2.3.4 Humidifier

Delivery of a flow of air without humidification may cause drying of airways. The use of a humidifier 5000 with an RPT device 4000 and the patient interface 3100 produces humidified gas that minimizes drying of the nasal mucosa and increases patient airway comfort. In addition, in cooler climates, warm air applied generally to the face area in and about the patient interface is more comfortable than cold air.

### 1.2.3.5 Vent Technologies

Some forms of treatment systems may include a vent to allow the washout of exhaled carbon dioxide. The vent 3400 may allow a flow of gas from an interior space of a patient interface. e.g., the plenum chamber, to an exterior of the patient interface, e.g., to ambient.

### 2 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

One form of the present technology comprises an air circuit for a respiratory therapy system, the air circuit comprising;
a conduit;
an electromagnet provided to at least a first end of the conduit, wherein at least a portion of the conduit is collapsible and/or compressible in an axial direction, wherein the portion is adjacent the electromagnet;
a helical wire provided to a wall of the collapsible and/or compressible portion of the conduit, wherein the helical wire comprises a magnetic material;
   wherein energising the electromagnet causes at least a portion of the helical wire adjacent the electromagnet to be attracted to the electromagnet, thereby reducing the length of the conduit.

In examples:
a. the magnetic material comprises a ferrous material;
b. the ferrous material comprises steel or iron-palladium;
c. the collapsible and/or compressible portion comprises a concertina structure;
d. the helical wire is embedded in a wall of the conduit; and/or
e. the electromagnet forms part of, or is integrated into, a connector for connecting the air circuit to a component of a respiratory therapy system;

Another form of the present technology comprises an air circuit for a respiratory therapy system, the air circuit comprising:
a flexible, collapsible and/or compressible conduit: and
a plurality of magnetic elements provided to a wall of the conduit, wherein the elements are spaced apart in an axial direction;
   wherein each magnetic element is configured to attract an adjacent magnetic element when the magnetic element and the adjacent magnetic element are less than a predetermined distance apart.

Another form of the present invention comprises an air circuit for a respiratory therapy system, the air circuit comprising a conduit, wherein at least a portion of the conduit is collapsible and/or compressible in an axial direction, the air circuit further comprising a plurality of magnets spaced axially along the collapsible and/or compressible portion, wherein each magnetic element is configured to attract an adjacent magnetic element when the magnetic element and the adjacent magnetic element are less than a predetermined distance apart.

Another form of the present technology comprises an air circuit for a respiratory therapy system, the air circuit comprising:
a flexible conduit; and
a plurality of pairs of magnetic elements provided to a wall of the conduit, wherein the magnetic elements in each pair are provided to opposite sides of the conduit and wherein the pairs of elements are spaced apart in an axial direction,
   wherein each magnetic element is configured to attract one of an adjacent pair of magnetic elements when the magnetic element and the adjacent magnetic element are less than a predetermined distance apart.

Another aspect of one form of the present technology is a patient interface that is moulded or otherwise constructed with a perimeter shape which is complementary to that of an intended wearer.

An aspect of one form of the present technology is a method of manufacturing apparatus.

An aspect of certain forms of the present technology is a medical device that is easy to use, e.g. by a person who does not have medical training, by a person who has limited dexterity, vision or by a person with limited experience in using this type of medical device.

An aspect of one form of the present technology is a portable RPT device that may be carried by a person, e.g., around the home of the person.

An aspect of one form of the present technology is a patient interface that may be washed in a home of a patient, e.g., in soapy water, without requiring specialised cleaning equipment. An aspect of one form of the present technology is a humidifier tank that may be washed in a home of a patient, e.g., in soapy water, without requiring specialised cleaning equipment.

The methods, systems, devices and apparatus described may be implemented so as to improve the functionality of a processor, such as a processor of a specific purpose computer, respiratory monitor and/or a respiratory therapy apparatus. Moreover, the described methods, systems, devices and apparatus can provide improvements in the technological field of automated management, monitoring and/or treatment of respiratory conditions, including, for example, sleep disordered breathing.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 3 BRIEF DESCRIPTION OF THE DRAWINGS

The present technology is illustrated by way of example, and not by way of limitation, in the figures of the accompanying drawings, in which like reference numerals refer to similar elements including:

### 3.1 Respiratory Therapy Systems

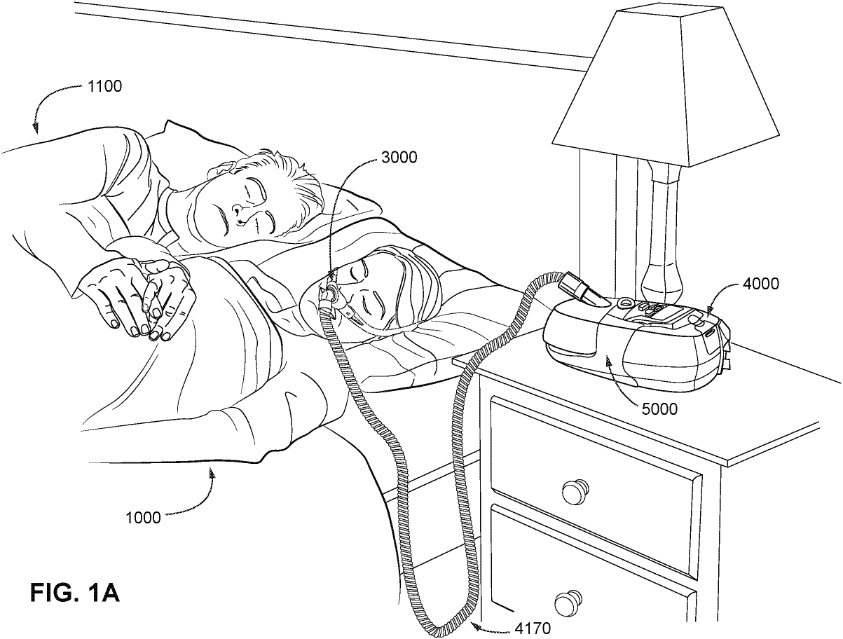

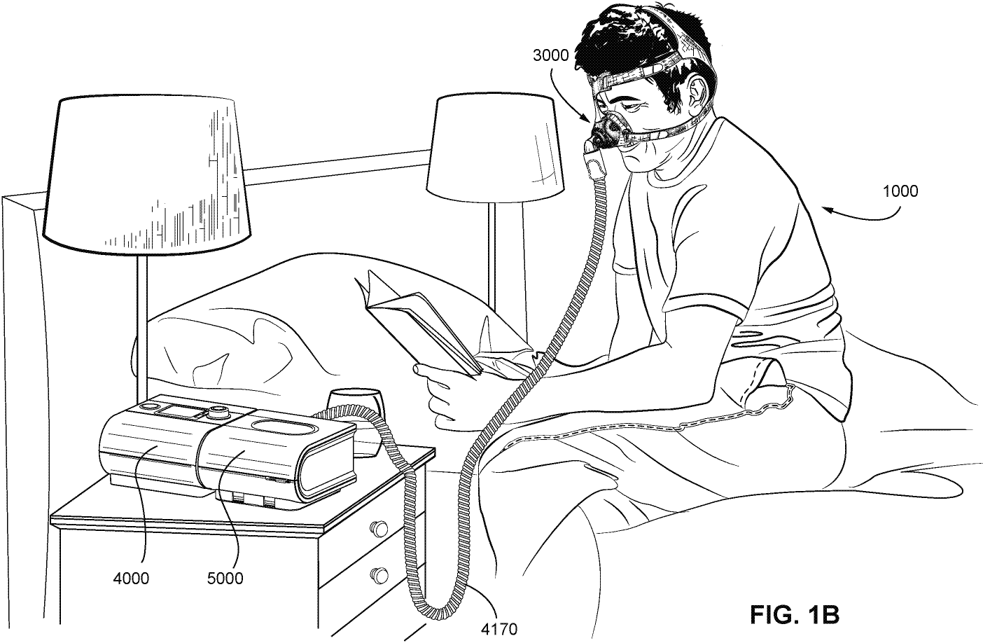

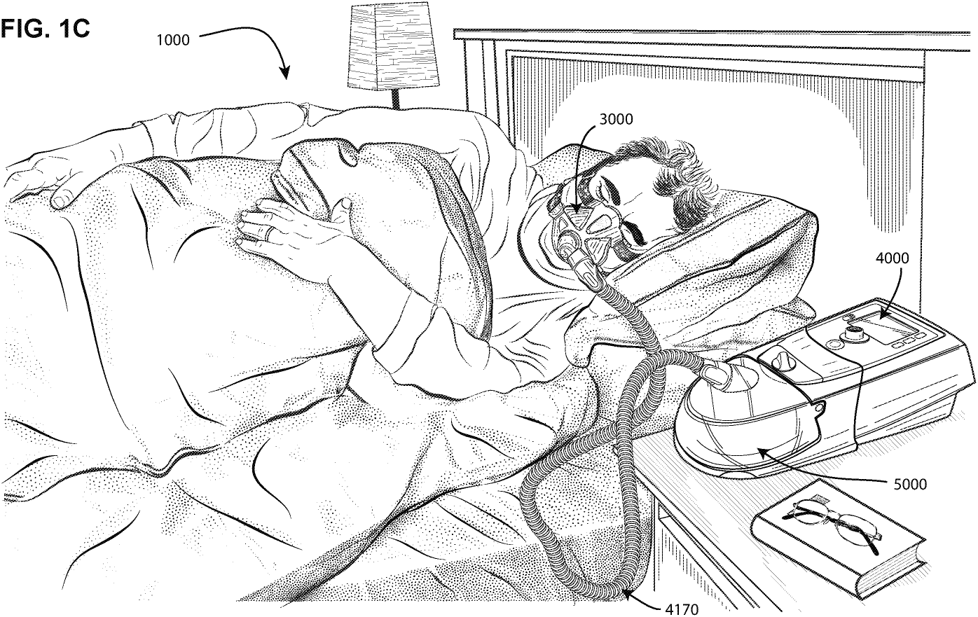

FIG. 1A shows a system including a patient 1000 wearing a patient interface 3000, in the form of nasal pillows, receiving a supply of air at positive pressure from an RPT device 4000. Air from the RPT device 4000 is humidified in a humidifier 5000. and passes along an air circuit 4170 to the patient 1000. A bed partner 1100 is also shown. The patient is sleeping in a supine sleeping position.

FIG. 1B shows a system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 1C shows a system including a patient 1000 wearing a patient interface 3000, in the form of a full-face mask, receiving a supply of air at positive pressure from an RPT device 4000. Air from the RPT device is humidified in a humidifier 5000. and passes along an air circuit 4170 to the patient 1000. The patient is sleeping in a side sleeping position.

### 3.2 Respiratory System and Facial Anatomy

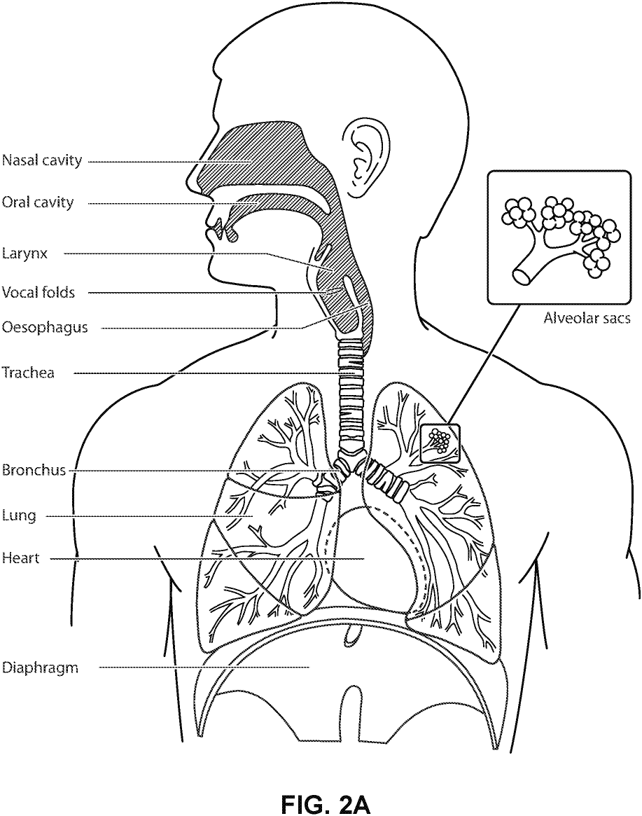

FIG. 2A shows an overview of a human respiratory system including the nasal and oral cavities, the larynx, vocal folds, oesophagus, trachea, bronchus, lung, alveolar sacs, heart and diaphragm.

### 3.3 Patient Interface

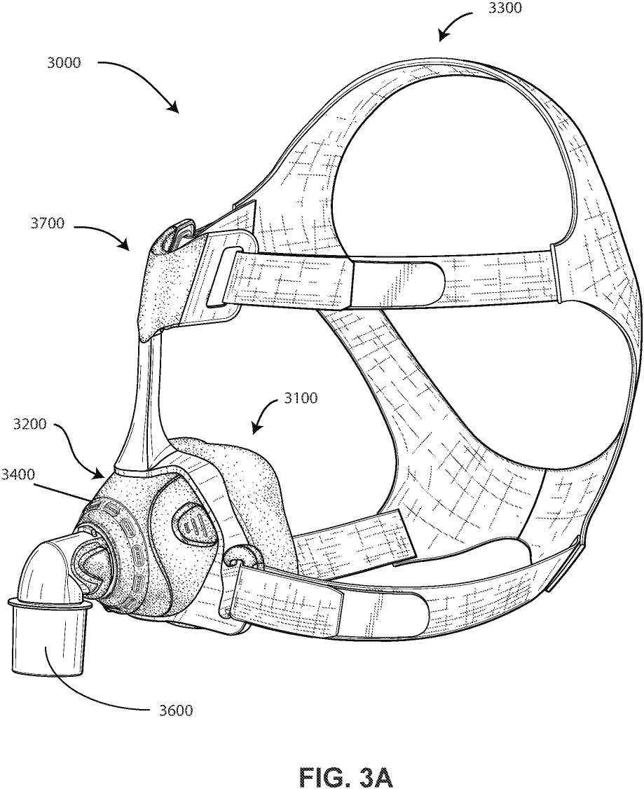

FIG. 3A shows a patient interface in the form of a nasal mask in accordance with one form of the present technology.

### 3.4 Air Circuits of the Present Technology

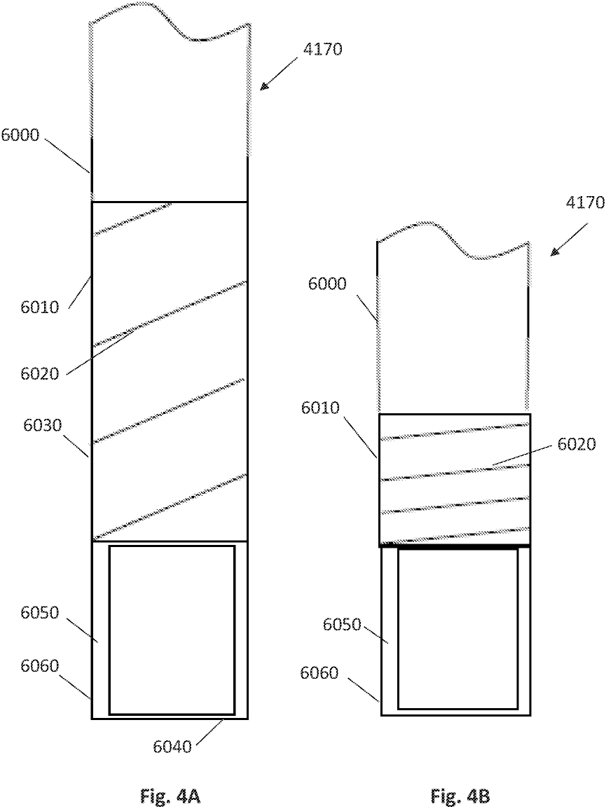

FIG. 4A shows, diagrammatically, a portion of an air circuit of one form of the technology.

FIG. 4B shows the portion of the air circuit shown in FIG. 4A with the electromagnet energised to reduce the length of the air circuit.

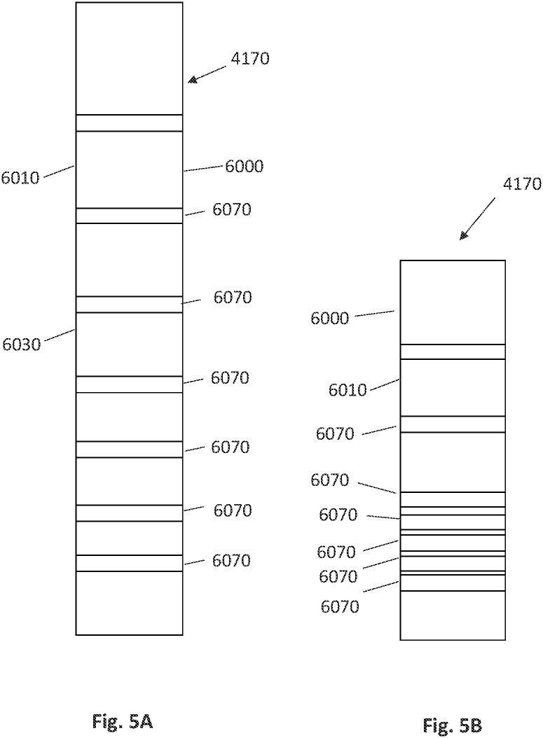

FIG. 5A shows, diagrammatically, a portion of an air circuit of one form of the technology.

FIG. 5B shows the portion of the air circuit of FIG. 5A in a reduced length configuration.

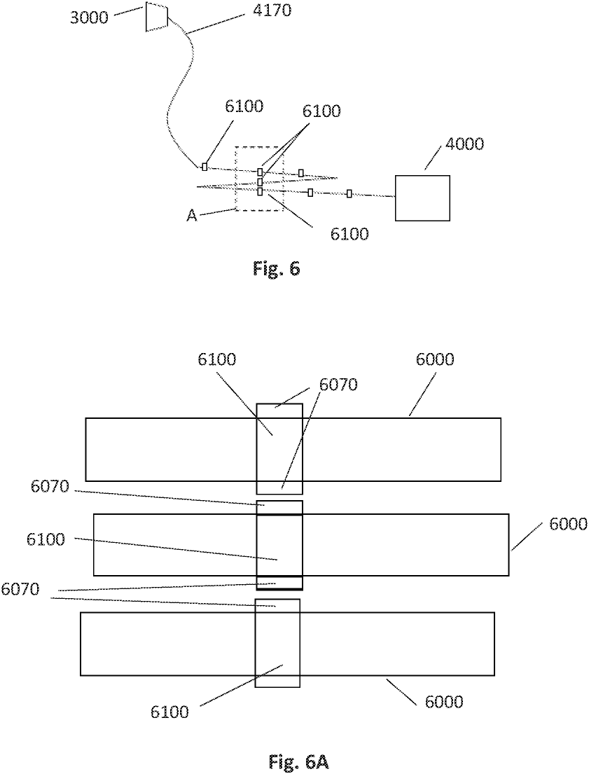

FIG. 6 shows, diagrammatically, a system for treatment of respiratory therapy according to one form of the technology.

FIG. 6A shows, diagrammatically, an enlargement of portion “A” of FIG. 6.

### 4 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 4.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 4.2 Respiratory Therapy Systems

In one form, the present technology comprises a respiratory therapy system for treating a respiratory disorder. The respiratory therapy system may comprise an RPT device 4000 for supplying a flow of air to the patient 1000 via an air circuit 4170 and a patient interface 3000.

### 4.3 Patient Interface

A non-invasive patient interface 3000, such as that shown in FIG. 3A, in accordance with one aspect of the present technology comprises the following functional aspects: a seal-forming structure 3100, a plenum chamber 3200, a positioning and stabilising structure 3300, a vent 3400, one form of connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

An unsealed patient interface, in the form of a nasal cannula (not shown), includes nasal prongs which can deliver air to respective nares of the patient 1000 via respective orifices in their tips. Such nasal prongs do not generally form a seal with the inner or outer skin surface of the nares. This type of interface results in one or more gaps that are present in use by design (intentional) but they are typically not fixed in size such that they may vary unpredictably by movement during use. This can present a complex pneumatic variable for a respiratory therapy system when pneumatic control and/or assessment is implemented, unlike other types of mask-based respiratory therapy systems. The air to the nasal prongs may be delivered by one or more air supply lumens that are coupled with the nasal cannula-type unsealed patient interface. The lumens lead from the nasal cannula-type unsealed patient interface to a respiratory therapy device via an air circuit. The unsealed patient interface is particularly suitable for delivery of flow therapies, in which the RPT device generates the flow of air at controlled flow rates rather than controlled pressures. The “vent” or gap at the unsealed patient interface, through which excess airflow escapes to ambient, is the passage between the end of the prongs of the nasal cannula-type unsealed patient interface via the patient’s nares to atmosphere.

### 4.4 RPT Device

An RPT device 4000 in accordance with one aspect of the present technology comprises mechanical, pneumatic, and/or electrical components and is configured to execute one or more algorithms, such as any of the methods, in whole or in part, described herein. The RPT device 4000 may be configured to generate a flow of air for delivery to a patient’s airways, such as to treat one or more of the respiratory conditions described elsewhere in the present document.

### 4.5 Air Circuit

An air circuit 4170 in accordance with an aspect of the present technology is a conduit or a tube constructed and arranged to allow, in use, a flow of air to travel between two components such as RPT device 4000 and the patient interface 3000.

The air circuit may be referred to as an air delivery tube. In some cases there may be separate limbs of the circuit for inhalation and exhalation. In other cases a single limb is used.

In some forms, the air circuit 4170 may comprise one or more heating elements configured to heat air in the air circuit, for example to maintain or raise the temperature of the air. The heating element may be in a form of a heated wire circuit, and may comprise one or more transducers, such as temperature sensors. In one form, the heated wire circuit may be helically wound around the axis of the air circuit 4170. The heating element may be in communication with a controller such as a central controller. One example of an air circuit 4170 comprising a heated wire circuit is described in U.S. Pat. 8,733,349, which is incorporated herewithin in its entirety by reference.

### 4.5.1 Adjustable Length Air Circuits

Referring next to FIGS. 4A and 4B, one form of the technology comprises an air circuit 4170 for a respiratory therapy system.

The air circuit 4170 comprise a conduit or tube 6000. At least a portion of the tube is collapsible and/or compressible in an axial direction. In examples the collapsible/compressible portion 6010 comprises a relatively thin wall. In examples the collapsible/compressible portion 6010 comprises a tube wall having a concertina structure.

The air circuit 4170 comprises a helical wire 6020 provided to the tube wall 6030. The helical wire 6020 may extend along at least the collapsible/ compressible portion 6010 of the tube 6000. In examples the helical wire 6020 may extend beyond the collapsible/compressible portion 6010, for example, along the entire length of the air circuit 4170. In examples where the helical wire 6020 does not extend along the entire length of the air circuit 4170, a reinforcing means, e.g. a moulded helical rib (not shown), may be provided to portions of the air circuit 4170 which the helical wire 6020 does not extend through.

The helical wire 6020 may be attached to an inner or an outer surface of the tube wall 6030, or may be embedded within the wall 6030 (for example during a moulding process). The helical wire 6020 may be ferrous, for example made from steel and/or iron-palladium. In other examples the helical wire may comprise an alternative magnetic material. As used herein, the term “magnetic material” refers to iron, amorphous iron based materials, or alloys of Ni-Fe, Co-Fe, Fe-Si, or ferrites based on at least one of manganese, zinc, nickel and magnesium and the like.

In examples the collapsible/compressible portion 6010 of the tube 6000 and the helical wire 6020 are adjacent one end 6040 of the tube 6000. An electromagnet 6050 is provided at the end of the tube. In examples, the electromagnet 6050 forms part of, or is integrated into, an air circuit connector 6060 for connecting the air circuit 4170 to another component of the therapy system (e.g an RPT device 4000 or a patient interface 3000). In examples, the connector 6060 conforms to ISO standard ISO 17510: 2015.

The electromagnet may be energised, in use, by a suitable power source (not shown). In examples, the power source may be integrated into the RPT.

As shown in FIG. 4B, when the electromagnet 6050 is activated, at least a portion of the helical wire 6020 is attracted towards the electromagnet 6050, thereby collapsing/compressing a corresponding part of the tube 6000, and reducing the tube’s overall effective length. The portion of the wire 6020 which is attracted to the electromagnet 6050 may be varied by varying the electrical current supplied to the electromagnet 6050 (and hence its magnetic strength). In this way the patient can select the length of the tube 6000, within certain limits, by controlling the electromagnet 6050.

When the current to the electromagnet 6050 is reduced or ceased, the tube 6000 may return to its original length. This restoring force may be as a result of resilience of the tube walls 6030 and/or due to the helical wire 6020 acting as a spring.

In examples, both ends of the air circuit 4170 may comprise electromagnets 6050 and corresponding collapsible/compressible portions 6010 and helical wire 6020 portions as described above. In this way the amount of adjustment possible may be increased. In examples one or more electromagnets and corresponding collapsible/compressible portions may be provided along the length of the air circuit,

In examples the entire length of the air circuit 4170 may be configured to be collapsible/compressible.

Referring next to FIG. 5A, in another form of the technology the air circuit 4170 comprises a conduit or tube 6000. At least a portion 6010 of the tube 6000 is collapsible and/or compressible. In examples the entire length of the tube 6000 is collapsible/compressible.

The collapsible/compressible portion 6010 of the tube 6000 is provided with a plurality of magnetic elements 6070. The magnetic elements 6070 are axially spaced apart, e.g. spaced apart along the length of the tube 6000. In examples the entire tube 6000 is collapsible/compressible and magnetic elements 6070 are spaced apart along the entire length of the tube 6000.

In examples the magnetic elements 6070 are configured as rings. The magnetic rings may be provided to an interior surface or an exterior surface of the tube, or may be embedded within the wall 6030 (for example during a moulding process).

The magnetic elements 6070 are configured such that each element 6070 attracts the immediately adjacent elements 6070, at least when there is less than a predetermined distance between the elements. The strength of the elements 6070, and the spacing between the elements 6070, may be selected such that when two elements 6070 are brought within the predetermined distance (for example by manipulation by the patient) the elements 6070 are attracted together and collapse the portion of the tube 6000 between them. However, the strength of the magnets (and the resilience of the tube) is selected such that the patient is able to separate them relatively easily, and such that any magnetic attraction between adjacent magnets when the tube 6000 is in an uncompressed state is not sufficient to collapse the tube 6000.

In this way, the patient can compress one or more portions of the tube 6000 to reduce the length of the tube 6000 (as shown in FIG. 5B) and the magnetic elements 6070 will keep the compressed portions in a compressed configuration. However, the patient can easily decompress any portion of the tube 6000 as required.

In examples, magnetic material elements may be interspersed between the magnetic elements, for example such that magnetic material elements and magnetic elements are provided alternately along the length of the conduit.

### 4.5.2 Configurable Tube

Referring next to FIGS. 6 and 6A. in another form of the technology a plurality of clips 6100 are provided. The clips 6100 are configured to releasably engage an exterior of the air circuit 4170.

Each clip 6100 comprises a pair of magnetic elements. The magnetic elements are arranged such that, in use, the elements in each pair are provided on opposite sides of the air circuit 4170.

Each magnetic element is configured to attract at least one magnetic element from an adjacent pair of magnetic elements, when the adjacent magnetic element is within a predetermined distance.

The clips 6100 can be used to keep the air circuit 4170 in a convenient configuration. For example, if the patient finds the air circuit 4170 to be too long, then a portion of the air circuit 4170 adjacent the RPT may be arranged in a coiled formation, with the clips operating to keep the air circuit 4170 in the required formation.

This form of the invention allows considerable flexibility in the routing of the air circuit 4170. For example, the air circuit 4170 may be coiled around the RPT to reduce its effective length. In examples, each clip 6100 may be placed at any location along the length of the air circuit 4170, to further improve the options for arranging the air circuit 4170.

The strength of the magnet elements is selected such that the patient is able to separate them relatively easily.

FIG. 6 shows a respiratory system comprising an RPT 4000 connected to a patient interface 3000 by an air circuit 4170. A plurality of clips 6100 are spaced apart along the air circuit 4170.

An intermediate portion of the air circuit 4170 is arranged in a coil and is held in this configuration by a plurality of the clips 6100.

### 4.6 Glossary

For the purposes of the present technology disclosure, in certain forms of the present technology, one or more of the following definitions may apply. In other forms of the present technology, alternative definitions may apply.

### 4.6.1 General

Air: In certain forms of the present technology, air may be taken to mean atmospheric air, and in other forms of the present technology air may be taken to mean some other combination of breathable gases, e.g. oxygen enriched air.

Ambient: In certain forms of the present technology, the term ambient will be taken to mean (i) external of the treatment system or patient and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

In the example of patient respiration, a flow rate may be nominally positive for the inspiratory portion of a breathing cycle of a patient, and hence negative for the expiratory portion of the breathing cycle of a patient. Device flow rate, Qd, is the flow rate of air leaving the RPT device. Total flow rate, Qt, is the flow rate of air and any supplementary gas reaching the patient interface via the air circuit. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient’s respiratory system.

Flow therapy: Respiratory therapy comprising the delivery of a flow of air to an entrance to the airways at a controlled flow rate referred to as the treatment flow rate that is typically positive throughout the patient’s breathing cycle.

Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient’s face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Oxygen enriched air: Air with a concentration of oxygen greater than that of atmospheric air (21%), for example at least about 50% oxygen, at least about 60% oxygen, at least about 70% oxygen, at least about 80% oxygen, at least about 90% oxygen, at least about 95% oxygen, at least about 98% oxygen, or at least about 99% oxygen. “Oxygen enriched air” is sometimes shortened to “oxygen”.

Medical Oxygen: Medical oxygen is defined as oxygen enriched air with an oxygen concentration of 80% or greater. Patient: A person, whether or not they are suffering from a respiratory condition.

Pressure: Force per unit area. Pressure may be expressed in a range of units, including cmH₂O, g-f/cm² and hectopascal. 1 cmH₂O is equal to 1 g-f/cm² and is approximately 0.98 hectopascal (1 hectopascal = 100 Pa = 100 N/m² = 1 millibar -0.001 atm). In this specification, unless otherwise stated, pressure is given in units of cmH₂O.

The pressure in the patient interface is given the symbol Pm, while the treatment pressure, which represents a target value to be achieved by the interface pressure Pm at the current instant of time, is given the symbol Pt.

Respiratory Pressure Therapy: The application of a supply of air to an entrance to the airways at a treatment pressure that is typically positive with respect to atmosphere.

Ventilator: A mechanical device that provides pressure support to a patient to perform some or all of the work of breathing.

### 4.6.1.1 Materials

Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45 as measured using ASTM D2240.

Polycarbonate: a thermoplastic polymer of Bisphenol-A Carbonate.

### 4.6.1.2 Mechanical Properties

Resilience: Ability of a material to absorb energy when deformed elastically and to release the energy upon unloading.

Resilient: Will release substantially all of the energy when unloaded. Includes e.g. certain silicones, and thermoplastic elastomers.

Hardness: The ability of a material per se to resist deformation (e.g. described by a Young’s Modulus, or an indentation hardness scale measured on a standardised sample size).
‘Soft’ materials may include silicone or thermo-plastic elastomer (TPE), and may, e.g. readily deform under finger pressure.
‘Hard’ materials may include polycarbonate, polypropylene, steel or aluminium, and may not e.g. readily deform under finger pressure.

Stiffness (or rigidity) of a structure or component: The ability of the structure or component to resist deformation in response to an applied load. The load may be a force or a moment, e.g. compression, tension, bending or torsion. The structure or component may offer different resistances in different directions. The inverse of stiffness is flexibility.

Floppy structure or component: A structure or component that will change shape, e.g. bend, when caused to support its own weight, within a relatively short period of time such as 1 second.

Rigid structure or component: A structure or component that will not substantially change shape when subject to the loads typically encountered in use. An example of such a use may be setting up and maintaining a patient interface in sealing relationship with an entrance to a patient’s airways, e.g. at a load of approximately 20 to 30 cmH2O pressure.

As an example, an I-beam may comprise a different bending stiffness (resistance to a bending load) in a first direction in comparison to a second, orthogonal direction. In another example, a structure or component may be floppy in a first direction and rigid in a second direction.

### 4.7 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

4.8 REFERENCE SIGNS LIST

| | |
|---|---|
| patient | 1000 |
| bed partner | 1100 |
| patient interface | 3000 |
| seal forming structure | 3100 |
| plenum chamber | 3200 |
| positioning and stabilizing structure | 3300 |
| vent | 3400 |
| connection port | 3600 |
| Forehead support | 3700 |
| RPT device | 4000 |
| air circuit | 4170 |
| Humidifier | 5000 |
| tube | 6000 |
| collapsible portion | 6010 |
| helical wire | 6020 |
| tube wall | 6030 |
| end | 6040 |
| electromagnet | 6050 |
| connector | 6060 |
| magnetic elements | 6070 |
| clips | 6100 |

1-11. (canceled)

12. An air circuit for a respiratory therapy system, the air circuit comprising;
a conduit;
an electromagnet provided to at least a first end of the conduit, wherein at least a portion of the conduit is collapsible and/or compressible in an axial direction, wherein the portion is adjacent the electromagnet; and
a helical wire provided to a wall of the collapsible and/or compressible portion of the conduit, wherein the helical wire comprises a magnetic material;
wherein energising the electromagnet causes at least a portion of the helical wire adjacent the electromagnet to be attracted to the electromagnet, thereby reducing the length of the conduit.

13. The air circuit of claim 12, wherein the magnetic material comprises a ferrous material.

14. The air circuit of claim 13, wherein the ferrous material comprises steel or iron-palladium.

15. The air circuit of claim 12, wherein the collapsible and/or compressible portion comprises a concertina structure.

16. The air circuit of claim 12, wherein the helical wire is embedded in a wall of the conduit.

17. The air circuit of claim 12, wherein the electromagnet forms part of, or is integrated into, a connector for connecting the air circuit to a component of a respiratory therapy system.

18. The air circuit of claim 17, wherein the component of a respiratory therapy system is an RPT device.

19. An air circuit for a respiratory therapy system, the air circuit comprising a conduit, wherein at least a portion of the conduit is collapsible and/or compressible in an axial direction, the air circuit further comprising a plurality of annular magnetic elements spaced axially along the collapsible and/or compressible portion, wherein each magnetic element is configured to attract an adjacent magnetic element when the magnetic element and the adjacent magnetic element are less than a predetermined distance apart.

20. The air circuit of claim 19, wherein the magnetic elements are embedded in a wall of the conduit.

21. An air circuit for a respiratory therapy system, the air circuit comprising:
a flexible conduit; and
a plurality of pairs of magnetic elements provided to a wall of the conduit, wherein the magnetic elements in each pair are provided to opposite sides of the conduit and wherein the pairs of elements are spaced apart in an axial direction,
wherein each magnetic element is configured to attract one of an adjacent pair of magnetic elements when the magnetic element and the adjacent magnetic element are less than a predetermined distance apart.

22. The air circuit of claim 21, wherein each pair of magnetic elements is provided to a respective clip.
